(19) United States
(12) Patent Application Publication
(10) Pub. No.: US 2017/0001957 A1
(43) Pub. Date: Jan. 5, 2017

(54) RADIOIODINATED BIOCONJUGATION REAGENTS

(71) Applicant: NUtech Ventures, Lincoln, NE (US)

(72) Inventors: Stephen DiMagno, Chicago, IL (US); Bao Hu, Lincoln, NE (US)

(73) Assignee: NUtech Ventures, Lincoln, NE (US)

(21) Appl. No.: 15/178,287

(22) Filed: Jun. 9, 2016

Related U.S. Application Data
(60) Provisional application No. 62/174,925, filed on Jun. 12, 2015.

(51) Int. Cl. C07D 207/46; A61K 51/04; C07D 207/452

(52) U.S. Cl. CPC C07D 207/46; C07D 207/452; A61K 51/0446

(57) ABSTRACT

This disclosure relates to radioiodinated compounds useful as bioconjugation reagents, and intermediates for making radioiodinated bioconjugation reagents.

### RELATED APPLICATION

This application claims priority to U.S. Provisional Application No. 62/174,925, filed on Jun. 12, 2015, the content of which is incorporated herein by reference in its entirety.

### STATEMENT OF FEDERALLY SPONSORED RESEARCH

This invention was made with government support under Grant No. R01EB015536 awarded by the National Institute of Biomedical Imaging and Bioengineering and National Institutes of Health. The Government has certain rights in the invention.

### TECHNICAL FIELD

This disclosure relates to radioiodinated compounds useful as bioconjugation reagents, and intermediates for making such compounds.

### BACKGROUND

A radiolabeled compound is a chemical compound comprising a radioactive atom; a radioiodinated compound comprises a radioactive isotope of iodine. Chemical and biological substrates may be radioiodinated for purposes including, for example, investigations of drug metabolism and tissue distribution. Bioconjugation refers to the formation of a covalent bond between a biological molecule and another chemical compound (a bioconjugation reagent. A radioiodinated bioconjugation reagent comprises a radioactive isotope of iodine and a reactive functional group (a “tag”) that selectively targets a specific residue of a biological molecule. There is a need for radioiodinated bioconjugation reagents, and intermediates for making such compounds.

### SUMMARY

Accordingly, the present application provides, inter alia, radioiodinated bioconjugation reagents and intermediates for making such compounds, collectively “the compounds of the invention.” In a first aspect, provided herein is a compound of formula (I):

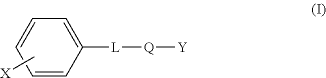

wherein X is selected from the group consisting of I-123, I-124, I-125 and I-131; L is an optionally substituted linker selected from the group consisting of alkyl, alkenyl, heteroalkyl, amidoalkyl, carboxyalkyl, and carboxyheteroalkyl; Q is absent, or is an optionally substituted linker selected from the group consisting of alkyl, heteroalkyl, aryl and carboxyalkyl; and Y is a bioconjugation tag selected from the group consisting of succinimidyl, sulfosuccinimidyl, maleimidyl, alkynyl, azide, isocyanate, isothiocyanate, iodoacetamide, 2-thiopyridonyl, 3-carboxy-4-nitrothiophenol, and diazobenzene.

In a second aspect, provided herein is a compound having the structure of formula (IV):

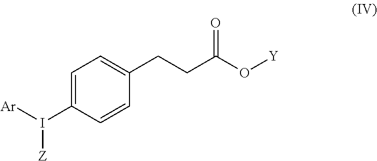

wherein Ar is an optionally substituted aryl or heteroaryl group; Z is an anion selected from the group consisting of halide, aryl carboxylate, alkyl carboxylate, phosphate, phosphonate, phosphonite, azide, thiocyanate, cyanate, phenoxide, arylsulfonate, alkylsulfonate, trifluoromethanesulfonate, thiolate, and stabilized enolate; and Y is a bioconjugation tag selected from the group consisting of succinimidyl and sulfosuccinimidyl.

In a third aspect, provided herein is a compound having the structure of formula (V):

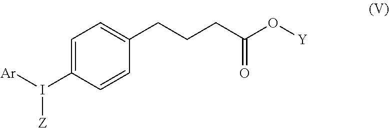

wherein Ar is an optionally substituted aryl or heteroaryl group; Z is an anion selected from the group consisting of halide, aryl carboxylate, alkyl carboxylate, phosphate, phosphonate, phosphonite, azide, thiocyanate, cyanate, phenoxide, arylsulfonate, alkylsulfonate, trifluoromethanesulfonate, thiolate, and stabilized enolate; and Y is a bioconjugation tag selected from the group consisting of succinimidyl and sulfosuccinimidyl.

### BRIEF DESCRIPTION OF THE DRAWINGS

FIG. 1 depicts the HPLC trace for the purification of the product of Example 3. HPLC method: C4 column, 1.25 mL/min, 20 mM ammonium acetate: MeOH (0% at 2 mins to 100% at 15 mins). The product elutes at 4 minutes.

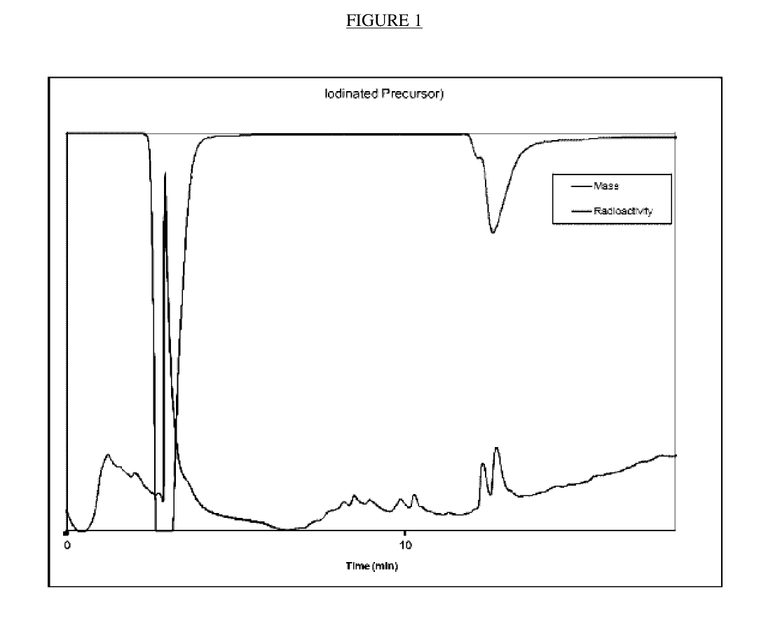

FIG. 2 depicts TLC data for Example 3. T LCs were eluted with a 50% EtOAc: hexanes solution.

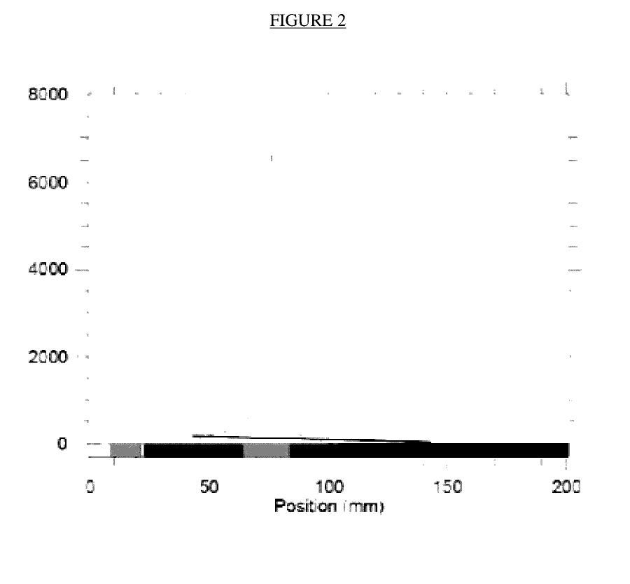

### DETAILED DESCRIPTION

The present application provides, inter alia, radioiodinated compounds useful as bioconjugation reagents, intermediates and methods for making said radioiodinated compounds.

### Compounds

In one aspect, provided herein is a compound of formula (I):

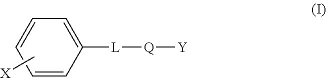

wherein X is selected from the group consisting of I-123, I-124, I-125 and I-131; L is an optionally substituted linker selected from the group consisting of alkyl, alkenyl, heteroalkyl, amidoalkyl, carboxyalkyl, and carboxyheteroalkyl; Q is absent, or is an optionally substituted linker selected from the group consisting of alkyl, heteroalkyl, aryl and carboxyalkyl; and Y is a bioconjugation tag selected from the group consisting of succinimidyl, sulfosuccinimidyl, maleimidyl, alkynyl, azide, isocyanate, isothiocyanate, iodoacetamide, 2-thiopyridonyl, 3-carboxy-4-nitrothiophenol, and diazobenzene.

In one embodiment, the compound of formula (I) has the structure of formula (II):

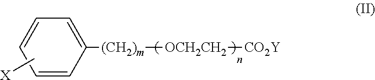

wherein m is 1-6; n is 0-3; and Y is selected from the group consisting of succinimidyl and sulfosuccinimidyl.

The compound of formula (II) may selected from group consisting of:

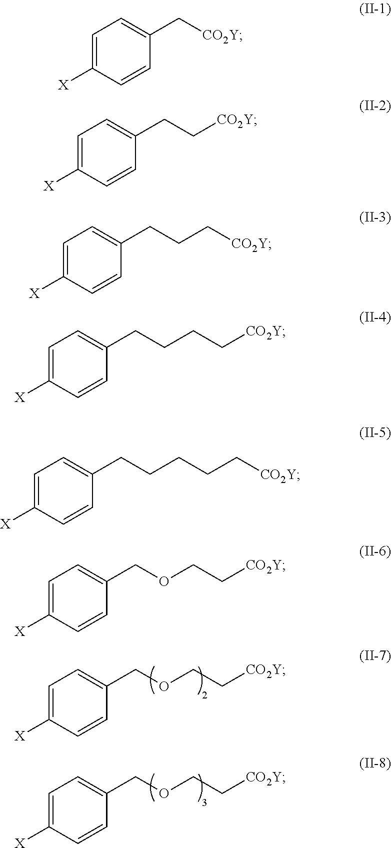

wherein X is selected from the group consisting of I-123, I-124, I-125 and I-131.

Compounds (II-2) and (II-3) may be selected from the group consisting of:

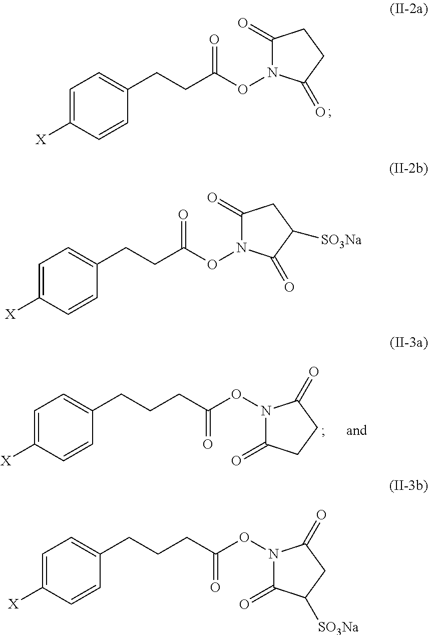

wherein X is selected from the group consisting of I-123, I-124, I-125 and I-131.

Compounds (II-2a), (II-2b), (II-3a) and (II-3b) may be selected from the group consisting of:

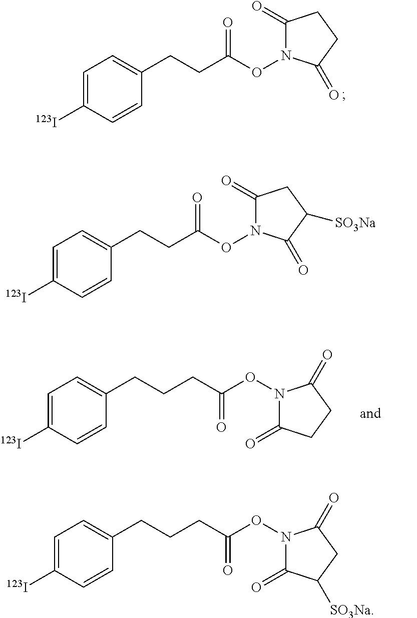

Compounds (II-2a), (II-2b), (II-3a) and (II-3b) may be selected from the group consisting of:

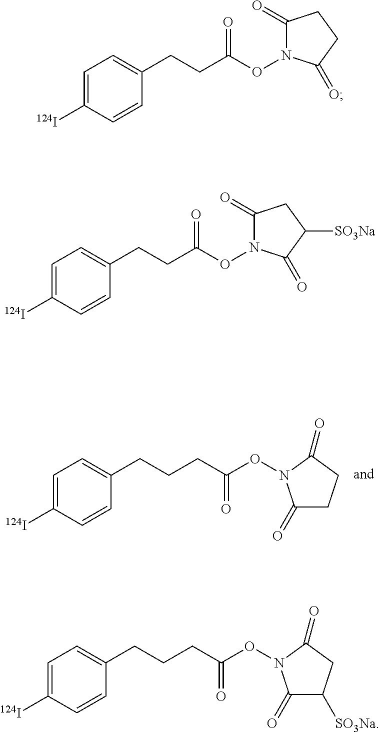

Compounds (II-2a), (II-2b), (II-3a) and (II-3b) may be selected from the group consisting of:

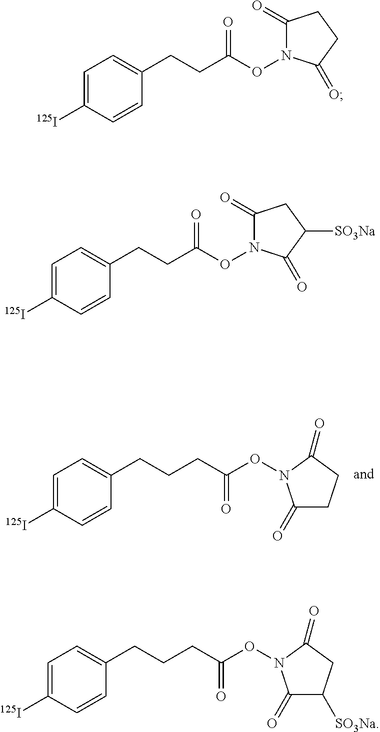

Compounds (II-2a), (II-2b), (II-3a) and (II-3b) may be selected from the group consisting of:

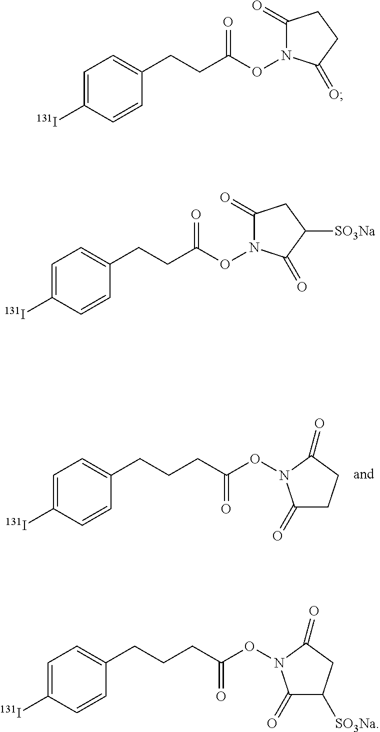

In another embodiment, the compound of formula (1) has the structure of formula (III):

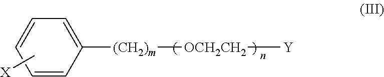

wherein m is 1-6; n is 0-3; and Y is selected from the group consisting of maleimidyl, azide, and alkynyl.

The compound of formula (III) may be selected from the group consisting of:

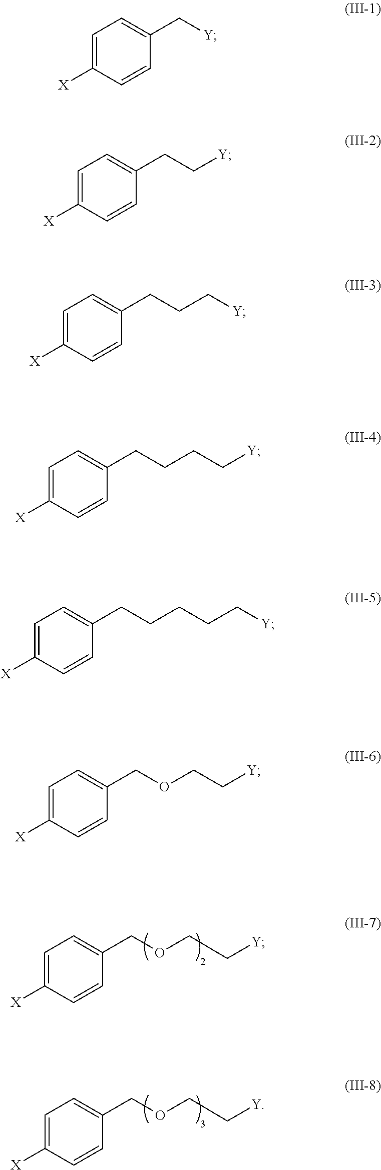

The compound of formula (III) may also be selected from the group consisting of:

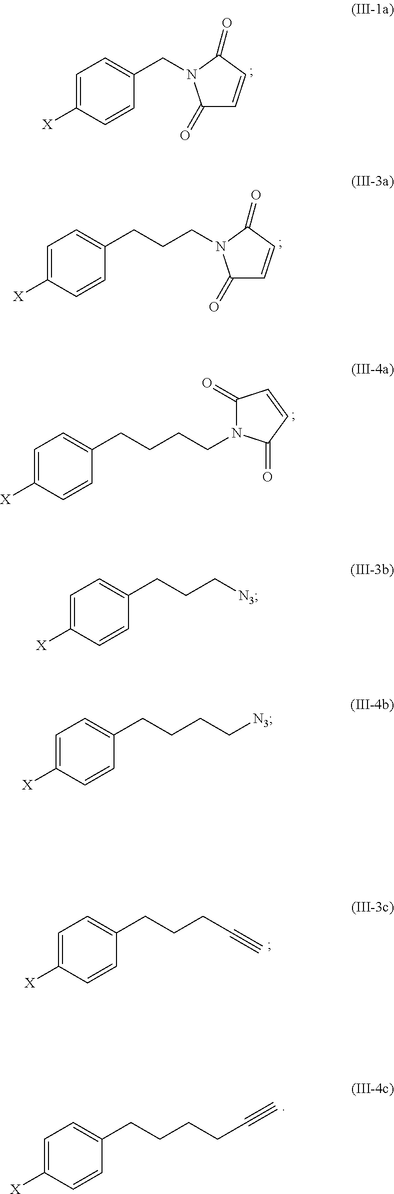

In another embodiment, the compound of formula (I) has the structure of formula (VI):

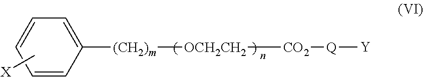

wherein m is 1-6; n is 0-3; Q is selected from alkyl, heteroalkyl, carboxyalkyl and carboxyheteroalkyl; and Y is selected from the group consisting of succinimidyl and sulfosuccinimidyl. In a particular embodiment of the compound of formula (VI), m is 2 or 3, and n is 0.

In another embodiment, the compound of formula (I) has the structure of formula (VII):

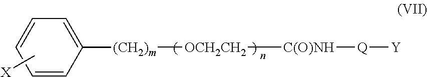

wherein m is 1-6; n is 0-3; Q is selected from alkyl, heteroalkyl, carboxyalkyl and carboxyheteroalkyl; and Y is selected from the group consisting of succinimidyl and sulfosuccinimidyl. In a particular embodiment of the compound of formula (VI), m is 2 or 3, and n is 0.

### Intermediates

In certain aspects, provided herein are intermediates in the synthesis of compounds of formula (I). In one aspect, provided herein is a compound having the structure of formula (IV):

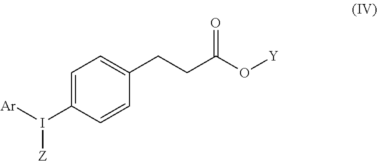

wherein Ar is an optionally substituted aryl or heteroaryl group; Z is an anion selected from the group consisting of halide, aryl carboxylate, alkyl carboxylate, phosphate, phosphonate, phosphonite, azide, thiocyanate, cyanate, phenoxide, arylsulfonate, alkylsulfonate, trifluoromethanesulfonate, thiolate, and stabilized enolate; and Y is a bioconjugation tag selected from the group consisting of succinimidyl and sulfosuccinimidyl.

In one embodiment, Ar is selected from the group consisting of substituted or unsubstituted phenyl. In another particular embodiment, Ar is phenyl substituted with alkoxy. In another particular embodiment, Ar is 4-methoxyphenyl.

The compound of formula (IV) may have the structure of formula (IV-1):

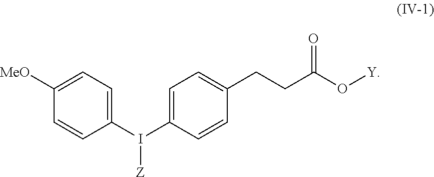

The compound of formula (IV) may have the structure of formula (IV-2):

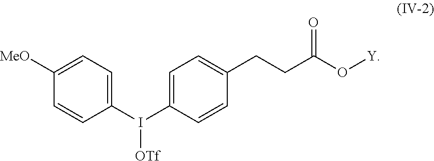

The compound of formula (IV-2) may have the structure of formula (IV-2a):

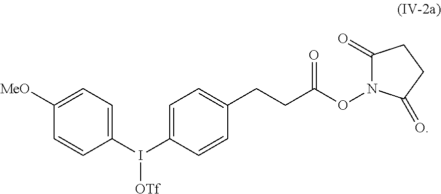 [SMILES: COc1ccc(I(OS(=O)(=O)C(F)(F)F)c2ccc(CCC(=O)ON3C(=O)CCC3=O)cc2)cc1]

The compound of formula (IV-2) may have the structure of formula (IV-2b):

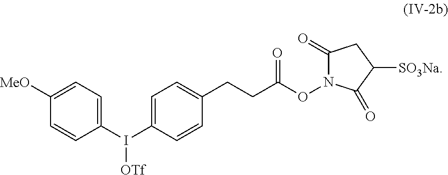

In another aspect, provided herein is a compound having the structure of formula (V):

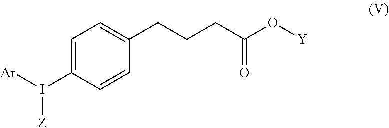

wherein Ar is an optionally substituted aryl or heteroaryl group; Z is an anion selected from the group consisting of halide, aryl carboxylate, alkyl carboxylate, phosphate, phosphonate, phosphonite, azide, thiocyanate, cyanate, phenoxide, arylsulfonate, alkylsulfonate, trifluoromethanesulfonate, thiolate, and stabilized enolate; and Y is a bioconjugation tag selected from the group consisting of succinimidyl and sulfosuccinimidyl.

In one embodiment, Ar is selected from the group consisting of substituted or unsubstituted phenyl. In another particular embodiment, Ar is phenyl substituted with alkoxy. In another particular embodiment, Ar is 4-methoxyphenyl.

The compound of formula (V) may have the structure of formula (V-1):

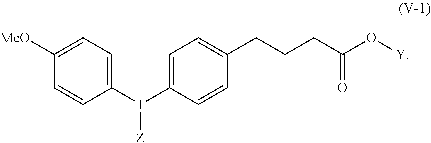

The compound of formula (V) may have the structure of formula (V-2):

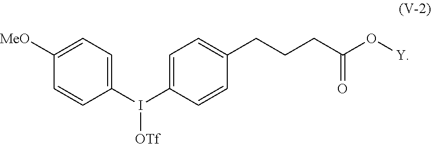

The compound of formula (V-2) may have the structure of formula (V-2a):

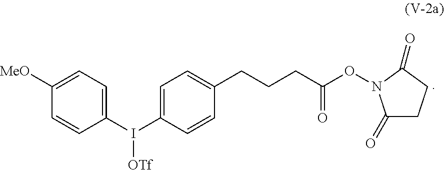 [SMILES: COc1ccc(I(OS(=O)(=O)C(F)(F)F)c2ccc(CCCC(=O)ON3C(=O)CCC3=O)cc2)cc1]

The compound of formula (V-2) may have the structure of formula (V-2b):

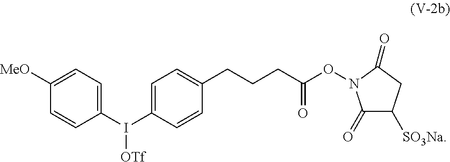

Also provided are compositions comprising a compound of formula (IV) and a compound of formula (II), or a compound of formula (V) and a compound of formula (III).

In one embodiment, provided herein is a composition comprising compound (IV-2a) and compound (II-2a):

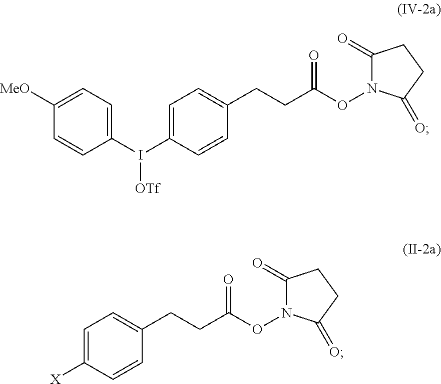

wherein X is selected from the group consisting of I-123, I-124, I-125 and I-131.

In another embodiment, provided herein is a composition comprising compound (IV-2b) and compound (II-2b):

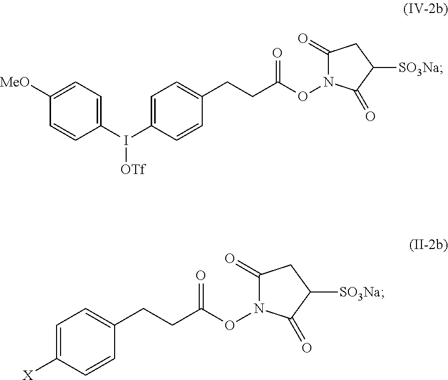

wherein X is selected from the group consisting of I-123, I-124, I-125 and I-131.

In another embodiment, provided herein is a composition comprising compound (V-2a) and compound (II-3a):

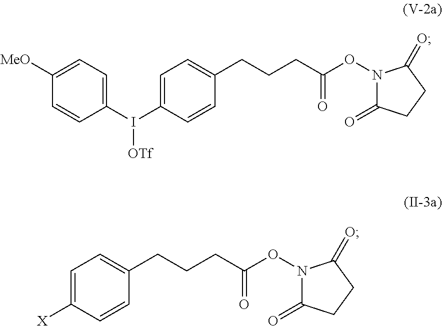

wherein X is selected from the group consisting of I-123, I-124, I-125 and I-131.

In another embodiment, provided herein is a composition comprising compound (V-2b) and compound (II-3b):

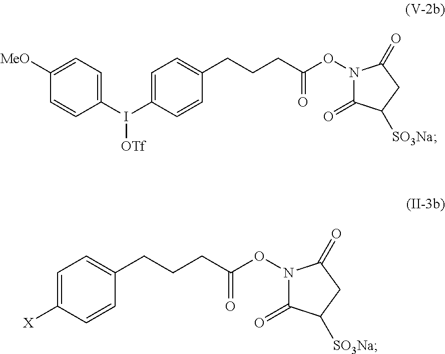

wherein X is selected from the group consisting of I-123, I-124, I-125 and I-131.

### Definitions

As used herein, the term “alkyl” refers to a fully saturated branched or unbranched hydrocarbon moiety. Preferably the alkyl comprises 1 to 20 carbon atoms, 1 to 16 carbon atoms, 1 to 10 carbon atoms, more preferably 1 to 6 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. Furthermore, the expression “Cₓ-C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl. The term “alkyl carboxylate” (e.g., acetate) refers to an alkyl group that is covalently bonded to the carbonyl carbon of a carboxylate moiety. An alkyl carboxylate may further comprise a counterion (e.g., a cation). The “alkylaryl” (e.g., benzyl) refers to an alkyl group that is covalently bonded to an aryl group, as aryl is defined below.

The term “alkenyl,” alone or in combination refers to a straight-chain, cyclic or branched hydrocarbon residue comprising at least one olefinic bond and the indicated number of carbon atoms. Preferred alkenyl groups have up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl, isobutenyl, 1-cyclohexenyl, 1-cyclopentenyl.

The term “alkynyl” includes unsaturated aliphatic groups analogous in length to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

As used herein, the term “cycloalkyl” refers to saturated or unsaturated monocyclic, bicyclic or tricyclic hydrocarbon groups of 3-12 carbon atoms, preferably 3-9, or 3-7 carbon atoms. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

The term “cycloalkenyl” refers to a partially unsaturated cyclic hydrocarbon group containing 1 to 3 rings and 4 to 8 carbons per ring. Exemplary groups include cyclobutenyl, cyclopentenyl, and cyclohexenyl. The term “cycloalkenyl” also includes bicyclic and tricyclic groups in which at least one of the rings is a partially unsaturated, carbon-containing ring and the second or third ring may be carbocyclic or heterocyclic, provided that the point of attachment is to the cycloalkenyl group.

“Alkoxy” refers to those alkyl groups, having from 1 to 10 carbon atoms, attached to the remainder of the molecule via an oxygen atom. Alkoxy groups with 1-8 carbon atoms are preferred. The alkyl portion of an alkoxy may be linear, cyclic, or branched, or a combination thereof. Examples of alkoxy groups include methoxy, ethoxy, isopropoxy, butoxy, cyclopentyloxy, and the like. An alkoxy group can also be represented by the following formula: —ORⁱ, where Rⁱ is the “alkyl portion” of an alkoxy group.

The term “heteroalkyl,” by itself or in combination with another term, means, unless otherwise stated, a stable straight or branched chain, or combinations thereof, consisting of the stated number of carbon atoms and from one to five heteroatoms, more preferably from one to three heteroatoms, selected from the group consisting of O, N, Si and S, and wherein the nitrogen and sulfur atoms may optionally be oxidized and the nitrogen heteroatom may optionally be quaternized. The heteroalkyl group is attached to the remainder of the molecule through a carbon atom or a heteroatom.

As used herein, the term “amidoalkyl” refers to an alkyl group, as defined above, bonded to the moiety —C(═O)NH— via either the carbon or the nitrogen (i.e., alkyl-C(═O)NH— or —NH(C═O)-alkyl).

As used herein, the term “amidoheteroalkyl” refers to a heteroalkyl group, as defined above, bonded to the moiety —C(═O)NH— via either the carbon or the oxygen (i.e., alkyl-C(═O)NH— or —NH(C═O)-alkyl).

As used herein, the term “carboxyalkyl” refers to an alkyl group, as defined above, bonded to the moiety —C(═O)O— via either the carbon or the oxygen (i.e., alkyl-C(═O)O— or —O(C═O)-alkyl).

As used herein, the term “carboxyheteroalkyl” refers to a heteroalkyl group, as defined above, bonded to the moiety —C(═O)O— via either the carbon or the oxygen (i.e., alkyl-C(═O)O— or —O(C═O)-alkyl).

The term “alkylcarbonyl” refers to a group having the formula —C(O)—Rⁱⁱ, wherein Rⁱⁱ is an alkyl group as defined above and wherein the total number of carbon atoms refers to the combined alkyl and carbonyl moieties. An “alkylcarbonyl” group can be attached to the remainder of the molecule via an alkyl group (i.e., -alkyl-C(O)—Rⁱⁱ).

The term “alkoxycarbonyl” refers to a group having the formula —C(O)O—Rⁱⁱⁱ, wherein Rⁱⁱⁱ is an alkyl group as defined above and wherein the total number of carbon atoms refers to the combined alkyl and carbonyl moieties. An “alkoxycarbonyl” group can be attached to the remainder of the molecule via an alkyl group (i.e., -alkyl-C(O)O—Rⁱⁱⁱ).

The term “heteroalkylcarbonyl” refers to a group having the formula —C(O)R^{iv}, wherein R^{iv} is a heteroalkyl group as defined above and wherein the total number of carbon atoms refers to the combined alkyl and carbonyl moieties. A “heteroalkylcarbonyl” group can be attached to the remainder of the molecule via an alkyl or heteroalkyl group (i.e., -alkyl-C(O)O—R^{iv} or -heteroalkyl-C(O)O—R^{iv}).

The term “aryl” includes aromatic monocyclic or multicyclic e.g., tricyclic, bicyclic, hydrocarbon ring systems consisting only of hydrogen and carbon and containing from six to nineteen carbon atoms, or six to ten carbon atoms, where the ring systems may be partially saturated. Aryl groups include, but are not limited to, groups such as phenyl, tolyl, xylyl, anthryl, naphthyl and phenanthryl. Aryl groups can also be fused or bridged with alicyclic or heterocyclic rings which are not aromatic so as to form a polycycle (e.g., tetralin). The term “aryl carboxylate” (e.g., benzoate) refers to an aryl group that is covalently bonded to the carbonyl carbon of a carboxylate moiety. An aryl carboxylate may further comprise a counterion (e.g., a cation).

The term “heteroaryl,” as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycloalkyl” refers to a five-member to ten-member, fully saturated or partially unsaturated nonaromatic heterocylic groups containing at least one carbon and at least one heteroatom such as O, S or N. The most frequent examples are piperidinyl, morpholinyl, piperazinyl, pyrrolidinyl or pirazinyl. Attachment of a heterocycloalkyl substituent can occur via a carbon atom or via a heteroatom.

Moreover, the alkyl, alkenyl, cycloalkyl, cycloalkenyl, alkoxy, aryl, heteroaryl, and heterocycloalkyl groups described above can be “unsubstituted” or “substituted.” The term “substituted” is intended to describe moieties having substituents replacing a hydrogen on one or more atoms, e.g. C, O or N, of a molecule. Such substituents can independently include, for example, one or more of the following: straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group.

The term “amine” or “amino” should be understood as being broadly applied to both a molecule, or a moiety or functional group, as generally understood in the art, and may be primary, secondary, or tertiary. The term “amine” or “amino” includes compounds where a nitrogen atom is covalently bonded to at least one carbon, hydrogen or heteroatom. The terms include, for example, but are not limited to, “alkyl amino,” “acylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

In a particular embodiment of the invention, the term “amine” or “amino” refers to substituents of the formulas N(R⁸)R⁹, CH₂N(R⁸)R⁹ and CH(CH₃)N(R⁸)R⁹, wherein R⁸ and R⁹ are each, independently, selected from the group consisting of H and (C₁-C₄-alkyl)₀₋₁G, wherein G is selected from the group consisting of COOH, H, PO_{S}H, SO₃H, Br, Cl, F, O—C₁₋₄-alkyl, S—C₁₋₄-alkyl, aryl, C(O)OC₁-C₆-alkyl, C(O)C₁-C₄-alkyl-COOH, C(O)C₁-C₄-alkyl and C(O)-aryl.

The description of the disclosure herein should be construed in congruity with the laws and principals of chemical bonding. For example, it may be necessary to remove a hydrogen atom in order accommodate a substitutent at any given location. Furthermore, it is to be understood that definitions of the variables (i.e., “R groups”), as well as the bond locations of the generic formulae of the invention (e.g., Formulas (I)-(X)), will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds.

The compounds of this invention may include asymmetric carbon atoms. It is to be understood accordingly that the isomers arising from such asymmetry (e.g., all enantiomers, stereoisomers, rotamers, tautomers, diastereomers, or racemates) are included within the scope of this invention. Such isomers can be obtained in substantially pure form by classical separation techniques and by stereochemically controlled synthesis. Furthermore, the structures and other compounds and moieties discussed in this application also include all tautomers thereof. Compounds described herein may be obtained through art recognized synthesis strategies.

It will also be noted that the substituents of some of the compounds of this invention include isomeric cyclic structures. It is to be understood accordingly that constitutional isomers of particular substituents are included within the scope of this invention, unless indicated otherwise. For example, the term “tetrazole” includes tetrazole, 2H-tetrazole, 3H-tetrazole, 4H-tetrazole and 5H-tetrazole.

The term “protected”, as used herein in reference to a chemical moiety (e.g., a guanidine moiety), means that the particular functional moiety, or constituent atoms thereof (e.g., nitrogen atoms), is temporarily blocked so that a reaction can be carried out selectively at another reactive site in a multifunctional compound. Such temporary blocking is accomplished using a “protecting group”, i.e., a chemical moiety that is covalently bonded to the “protected” moiety or atom. Nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds.

### EXAMPLES

### Example 1

### Synthesis of [4-((2,5-Dioxo-2,5-dihydro-1H-pyrrol-1-yl)methyl)phenyl]-(4′-methoxyphenyl)iodonium triflate

### (1) N-(4-iodobenzyl) maleimide

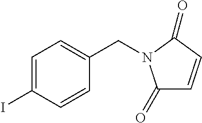 [SMILES: O=C1C=CC(=O)N1Cc1ccc(I)cc1]

DIAD (12 mmol, 2.43 g, 2.40 mL, 1.2 eq.) was added over the course of one hour to a solution of 4-iodobenzyl alcohol (10 mmol, 2.34 g, 1.0 eq.), PPh₃ (11 mmol, 2.88 g, 1.1 eq.), and maleimide (11 mmol, 1.07 g, 1.1 eq.) in 100 mL of THF. After the resulting yellow solution was stirred 12 h at room temperature, the solvent was removed by rotary evaporation and the residue was purified by column chromatography on silica gel (hexanes:ethyl acetate=1:5) and washed with hexane to obtain 2.00 g (64%) of N-(4-iodobenyl)maleimide as a colorless solid. ¹H NMR (CD₃CN, 400 MHz): δ 7.68 (d, J=8.4 Hz, 2H), 7.06 (d, J=8.4 Hz, 2H), 6.79 (s, 2H), 4.57 (s, 2H); 1H NMR (C₆D₆, 400 MHz): d 7.35 (d, J=8.0 Hz, 2H), 6.81 (d, J=8.4 Hz, 2H), 5.61 (s, 2H), 4.13 (s, 2H); ¹³C NMR (100 MHz, CD₃CN) δ 169.1, 136.0, 135.2,132.8, 128.1, 90.7, 38.7; HRMS (EI) Calcd for C₁₁H₈NO₂I (M)⁺: 312.9600; Found: 312.9608.

### (2) [4-((2,5-Dioxo-2,5-dihydro-1H-pyrrol-1-yl)methyl)phenyl]-(4′-methoxyphenyl)iodonium triflate

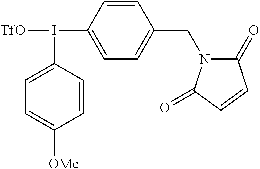 [SMILES: COc1ccc(I(OS(=O)(=O)C(F)(F)F)c2ccc(CN3C(=O)C=CC3=O)cc2)cc1]

In a N₂ charged glovebox, a solution of TMSOAc (7.8 mmol, 1.03 g, 2.6 eq.) in 50 mL of dry CH3CN was added dropwise to a solution of Selectfluor™ (3.9 mmol, 1.38 g, 1.3 eq.) in 50 mL of dry CH3CN. The resulting colorless mixture was then added dropwise to a solution of N-(4-iodobenzyl)maleimide (3 mmol, 0.94 g, 1.0 eq.) in dry CH₃CN (150 mL). After the resulting solution was stirred at room temperature for two days, potassium 4-methoxyphenyltrifluoroborate (0.64 g, 3 mmol, 1.0 equiv.) was added. Immediately thereafter, a solution of TMSOTf (0.53 g, 2.4 mmol, 0.8 eq.) in 50 mL of dry CH₃CN was added in a dropwise fashion, and the mixture was allowed to stand at room temperature for 30 min. The acetonitrile was removed under reduced pressure. Deionized water (200 mL) was added to the remaining solid and the mixture was extracted (3×50 mL) with CH₂Cl₂. The combined organic layers were washed with water (50 mL) and the obtained water layer was extracted (3×50 mL) with CH₂Cl₂ again. The combined organic extracts were dried over sodium sulfate, filtered, and the solvent was removed by rotary evaporation. The remaining solid was dissolved in 1 mL acetonitrile/water (9: 1 by volume) solution and slowly passed down an Amberlite IRA-400 ion exchange column, that was previously exchanged to the triflate counterion. After removal of the solvents under reduced pressure, the diaryliodonium triflate product was purified by washing the colorless residue with EtOAc to remove any organic impurities. The diaryliodonium salt was isolated as a colorless solid (0.91 g, 53%)). ¹H NMR (CD₃CN, 400 MHz): δ 7.99 (d, J=9.2 Hz, 2H), 7.97 (d, J=8.8 Hz, 2H), 7.40 (d, J=8.4 Hz, 2H), 7.05 (d, J=9.2 Hz, 2H), 6.81 (s, 2H), 4.67 (s, 2H), 3.84 (s, 3H); ¹³C NMR (100 MHz, CD₃CN) δ 169.0, 161.7, 140.6, 136.1, 133.5, 132.9, 129.6, 116.5, 111.0, 100.0, 54.1, 38.6; ¹⁹F NMR (CD₃CN, 376 MHz): δ −79.3 (s, 3F). HRMS (ESI) Calcd for C₁₈H₁₅NO₃I (M-OTf)⁺: 420.0097; Found: 420.0078.

### Example 2

### Synthesis of Propanoic Acid Succinimidyl Ester Diaryliodonium Salt (V-2a)

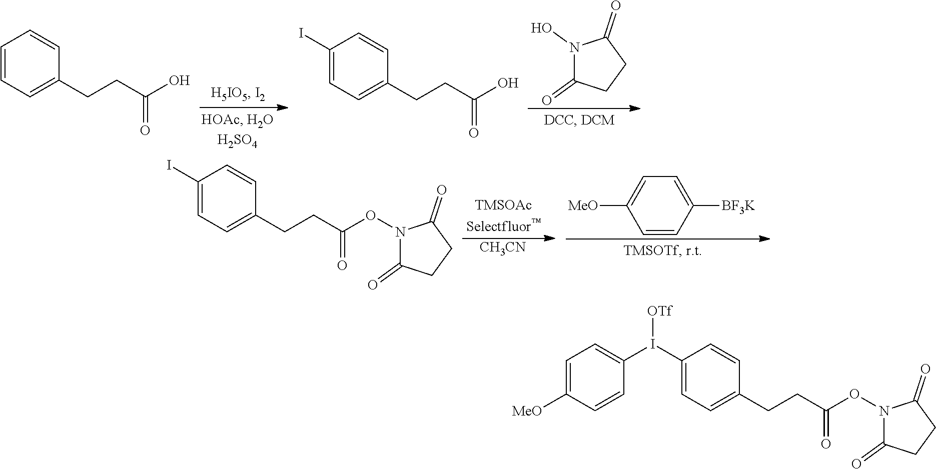

### (1) 3-(4′-Iodophenyl)propanoic Acid

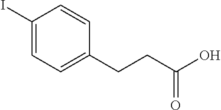 [SMILES: O=C(O)CCc1ccc(I)cc1]

A mixture of 3-phenylpropanoic acid (6.00 g, 40.0 mmol), H₅IO₆ (2.00 g, 8.60 mmol), iodine (4.06 g, 16.0 mmol) and 98% H₂SO₄ (1.2 mL) in water (8 mL) and acetic acid (40 mL) was heated at 67° C. for 17 h. The reaction mixture was cooled and quenched with water (100 mL). The crude product was then filtered, washed with water and hexane. The pure product (6.2 g, 56%) was obtained by recrystallization from toluene. White solid. ¹H NMR (400 MHz, CDCl₃) δ 7.62 (d, J=8.4 Hz, 2H), 6.97 (d, J=8.0 Hz, 2H), 2.91 (t, J=7.6 Hz, 2H), 2.67 (t, J=7.6 Hz, 2H).

### (2) 3-(4-Iodophenyl)propanoic Acid Succinimidyl Ester

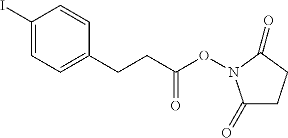 [SMILES: O=C(CCc1ccc(I)cc1)ON1C(=O)CCC1=O]

3-(4′-Iodophenyl)propanoic acid (10 mmol, 2.76 g, 1.0 eq.) and N-hydroxysuccinimide (15 mmol, 1.73 g, 1.5 eq) were dissolved in anhydrous CH₂Cl₂ (40 mL). The mixture was cooled to 0° C., and N,N′-dicyclohexylcarbodiimide (DCC, 15 mmol, 3.09 g, 1.5 eq) dissolved in 20 mL CH₂Cl₂ was added dropwise slowly. The mixture was stirred overnight at room temperature. The N,N′-dicyclohexylurea was filtered out, the residue was washed with CH₂Cl₂, and the filtrate was evaporated to dryness purified by column chromatography and recrystallization with iso-propanol or toluene/hexane to afford succinimide as white solid (3.50 g, 94%). ¹HNMR (400 MHz, CD₃CN) δ 7.66 (d, J=8.4 Hz, 2H), 7.08 (d, J=8.4 Hz, 2H), 2.95 (A₂B₂, t, 4H), 2.75 (s, 4H). ¹³CNMR (100 MHz, CD₃CN) δ 168.4, 166.7, 138.1, 135.9, 129.2, 89.5, 30.1, 27.8, 23.8; HRMS (ESI) Calcd for C₁₃H₁₂NO₄INa (M+Na)+: 395.9709; Found: 395.9706.

### (3) [4-(3-((2,5-dioxopyrrolidin-1-yl)oxy)-3-oxopropyl)phenyl]-(4′-methoxyphenyl)iodonium triflate (V-2a)

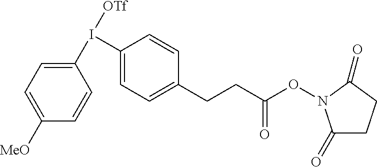 [SMILES: COc1ccc(I(OS(=O)(=O)C(F)(F)F)c2ccc(CCC(=O)ON3C(=O)CCC3=O)cc2)cc1]

In a N₂ charged glovebox, a solution of TMSOAc (13.0 mmol, 1.72 g, 2.6 eq.) in 20 mL dry CH₃CN was added dropwise to a solution of selectfluor™ (6.5 mmol, 2.30 g, 1.3 eq.) in 20 mL dry CH₃CN. The resulting colorless mixture was then added dropwise to a solution of (5.0 mmol, 1.87 g, 1.0 eq.) of 3-(4-Iodophenyl)propanoic acid succinimidyl ester in 20 mL dry CD₃CN. After being stirred at room temperature for 17 h, trifluoroborate salt (1.07 g, 5 mmol, 1.0 equiv.) was added to the solution followed by a solution of TMSOTf (1.00 g, 4.5 mmol, 0.9 eq.) in 20.0 mL of dry CH₃CN was added dropwise and the mixture was allowed to stand at room temperature for 30 min. After the solvent acetonitrile was removed, 100 mL of deionized water was added and the mixture was extracted (30 mL×3) with CH₂CH₂. The combined organic layers were washed with water (50 mL×1) and the obtained water layers were extracted (50 mL×2) with CH₂CH₂ again. The combined organic extracts were dried over sodium sulfate, filtered, and the solvent was removed by rotary evaporation. The residue was dissolved in 1.0 mL CH₃CN and added dropwise to 200 mL of MTBE to precipitate the diaryliodonium triflate product. This solid was dissolved in 1 mL acetonitrile/water (9:1 by volume) solution and slowly passed down an Amberlite IRA-400 ion exchange column (triflate counterion). After removal of the solvents under reduced pressure, the purified iodonium triflate product (2.49 g, 79%) of was obtained as a colorless solid. 1H NMR (400 MHz, CD3CN) δ 8.01 (d, J=9.2 Hz, 2H), 7.98 (d, J=8.4 Hz, 2H), 7.44 (d, J=8.4 Hz, 2H), 7.05 (d, J=9.2 Hz, 2H), 3.84 (s, 3H), 3.07 (t, J=6.8 Hz, 2H), 2.97 (t, J=6.8 Hz, 2H), 2.74 (s, 4H); 13C NMR (75 MHz, CD₃CN) δ 170.0, 168.2, 163.3, 145.4, 137.6, 135.1, 132.5, 118.1, 111.6, 101.6, 55.7, 31.3, 29.5, 25.4; 19F NMR (CD₃CN, 282 MHz): d −79.2 (s, 3F). HRMS (ESI) Calcd for C₂₀H₁₉NO₅I (M-OTf)+: 480.0308; Found: 480.0310.

### Example 3

### Iodination Stud with No¹²⁴I in 50% CH₃CN Toluene

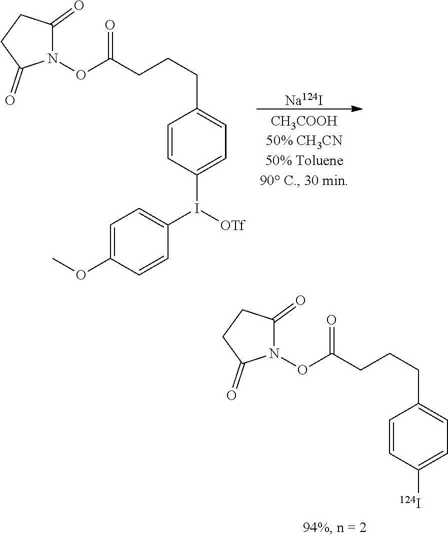 [SMILES: COc1ccc(I(OS(=O)(=O)C(F)(F)F)c2ccc(CCCC(=O)ON3C(=O)CCC3=O)cc2)cc1 ; "Na124I" ; O=C(CCCc1ccc([124I])cc1)ON1C(=O)CCC1=O]

Aqueous Na¹²⁴ I was dissolved in 0.1M NaOH. 1 μL of the Na¹²⁴I solution (approximately 1 mCi) was added to a first reaction vial along with 1 μL of 1.0 M AcOH to afford an acidic, slightly buffered solution. The initial activity was recorded for later calculations. Because the volume of water was so small, initial drying of the Na¹²⁴I solution was not required.

5 mg of the diaryliodonium precursor [4-(3-((2,5-dioxopyrrolidin-1-yl)oxy)-3-oxopropyl)phenyl]-(4′-methoxyphenyl)iodonium triflate was dissolved in 400 μL of CH₃CN. The mixture was allowed to stand for 10 minutes to make certain all of the crystalline compound had dissolved. The diaryliodonium precursor was added to the first reaction vial and then the solution was evaporated with a stream of dry argon at 90° C. After the solvent was removed completely, 125 μL of CH₃CN was added (with shaking or stirring) to dissolve the salts. Toluene (125 μL) was added and the solution was heated at 90° C. for 30 minutes. Silica TLC (100% ethyl acetate) was performed to determine the labeling efficiency. The reaction mixture was purified by passing it through a silica sep-pak and the crude product was purified by reverse phase HPLC to afford the desired product in 94% yield.

### Example 4

### General Procedure for the Radioiodination of NHS-Ester and Maleimide Conjugation Reagents

A diaryliodonium salt precursor (5 mg) was dissolved in 2.5 mL of dry acetonitrile. An aqueous solution of NaI (in which the isotope of iodide was either ¹²³I, ¹²⁴I, ¹²⁵I, or ¹³¹I) was counted and added to 400 μL of dry acetonitrile. If the initial NaI source was basic, 10 equivalents of acetic acid (per equivalent of NaOH) was added, and the solution was mixed and evaporated to dryness at 80° C. An aliquot of the precursor solution (400 μL) was added to the dried NaI and the resulting mixture was again evaporated to dryness. Dry acetonitrile (125 μL) was added to dissolve the residue, and 125 μL of dry toluene was added to adjust the solvent polarity. The reaction mixture was heated at 95° C. for 1 hour. Monitoring by radioTLC showed that radioiodide incorporation was 50-94% after 1 hour. Sep-pak purification was performed to remove the residual radioiodide and starting material. Final purification and/or quality control was performed by reverse phase HPLC.

1. A compound of formula (I): 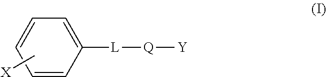
wherein X is selected from the group consisting of I-123, I-124, I-125 and 1-131;
L is an optionally substituted linker selected from the group consisting of alkyl, alkenyl, heteroalkyl, amidoalkyl, carboxyalkyl, and carboxyheteroalkyl;
Q is absent, or is an optionally substituted linker selected from the group consisting of alkyl, heteroalkyl, aryl and carboxyalkyl; and
Y is a bioconjugation tag selected from the group consisting of succinimidyl, sulfosuccinimidyl, maleimidyl, alkynyl, azide, isocyanate, isothiocyanate, iodoacetamide, 2-thiopyridonyl, 3-carboxy-4-nitrothiophenol, and diazobenzene.

2. The compound of claim 1 having the structure of formula (II): 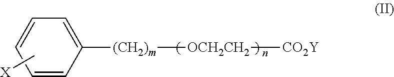
wherein m is 1-6;
n is 0-3; and
Y is selected from the group consisting of succinimidyl and sulfosuccinimidyl.

3. The compound of claim 2 selected from group consisting of:

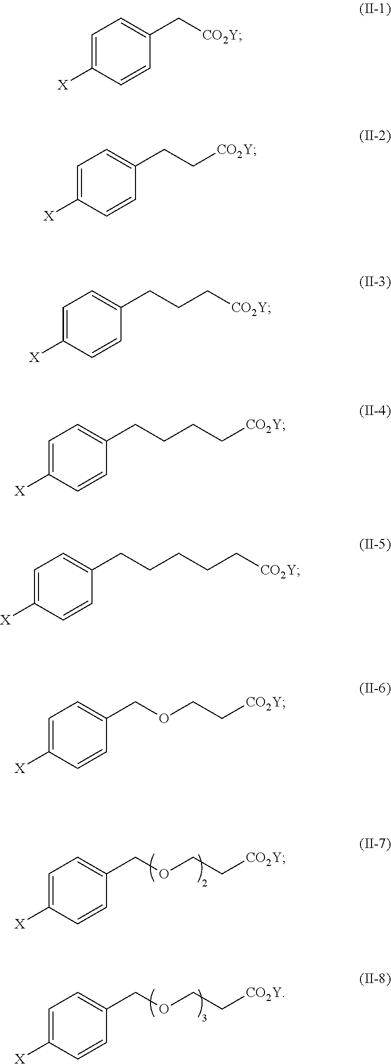

4-8. (canceled)

9. The compound of claim 1 having the structure of formula (III): 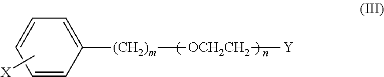
wherein m is 1-6;
n is 0-3; and
Y is selected from the group consisting of maleimidyl, azide, and alkynyl.

10. The compound of claim 9 selected from the group consisting of:

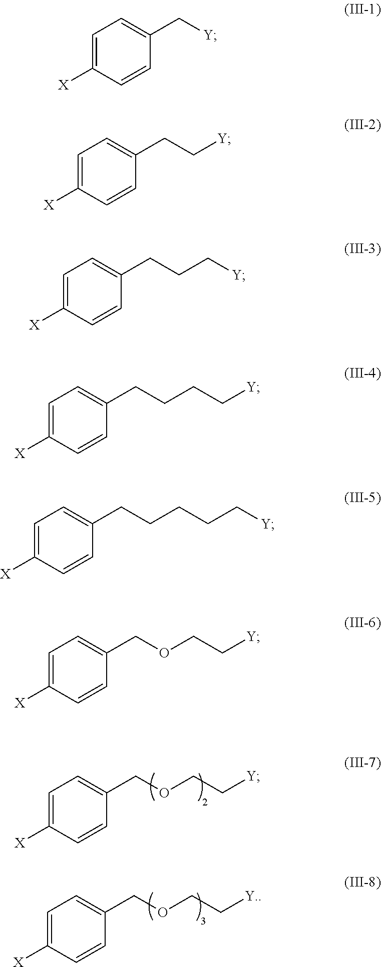

11. (canceled)

12. A compound having the structure of formula (IV): 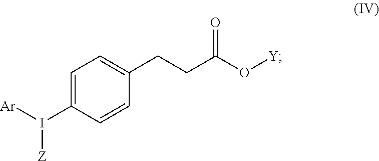
wherein Ar is an optionally substituted aryl or heteroaryl group;
Z is an anion selected from the group consisting of halide, aryl carboxylate, alkyl carboxylate, phosphate, phosphonate, phosphonite, azide, thiocyanate, cyanate, phenoxide, arylsulfonate, alkylsulfonate, trifluoromethanesulfonate, thiolate, and stabilized enolate; and
Y is a bioconjugation tag selected from the group consisting of succinimidyl and sulfosuccinimidyl.

13. (canceled)

14. The compound of claim 12 having the structure of formula (IV-2):

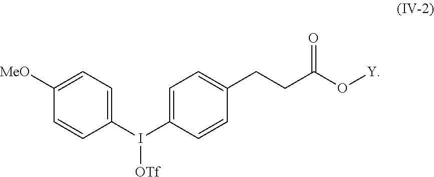

15. The compound of claim 14 having the structure of compound (IV-2a):

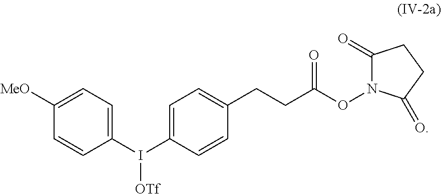 [SMILES: COc1ccc(I(OS(=O)(=O)C(F)(F)F)c2ccc(CCC(=O)ON3C(=O)CCC3=O)cc2)cc1]

16. The compound of claim 14 having the structure of compound (IV-2b):

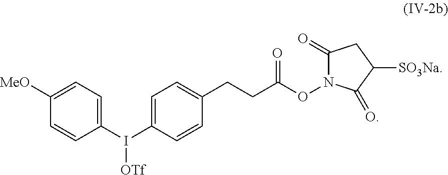

17. A composition comprising the compound of claim 15 and the compound (II-2a): 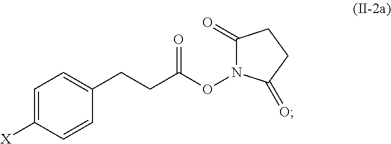
wherein X is selected from the group consisting of I-123, I-124, I-125 and I-131.

18. A composition comprising the compound of claim 16 and the compound (II-2b): 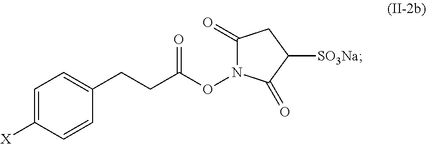
wherein X is selected from the group consisting of I-123, I-124, I-125 and I-131.

19. A compound having the structure of formula (V): 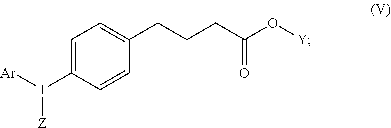
wherein Ar is an optionally substituted aryl or heteroaryl group;
Z is an anion selected from the group consisting of halide, aryl carboxylate, alkyl carboxylate, phosphate, phosphonate, phosphonite, azide, thiocyanate, cyanate, phenoxide, arylsulfonate, alkylsulfonate, trifluoromethanesulfonate, thiolate, and stabilized enolate; and
Y is a bioconjugation tag selected from the group consisting of succinimidyl and sulfosuccinimidyl.

20. The compound of claim 19 having the structure of formula (V-1):

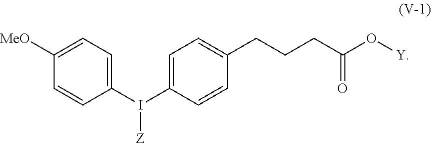

21. The compound of claim 19 having the structure of formula (V-2):

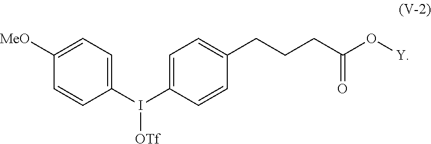

22. The compound of claim 21 having the structure of compound (V-2a):

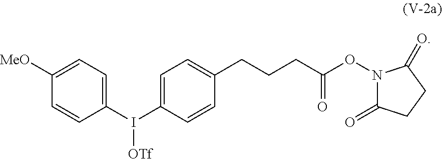 [SMILES: COc1ccc(I(OS(=O)(=O)C(F)(F)F)c2ccc(CCCC(=O)ON3C(=O)CCC3=O)cc2)cc1]

23. The compound of claim 21 having the structure of compound (V-2b):

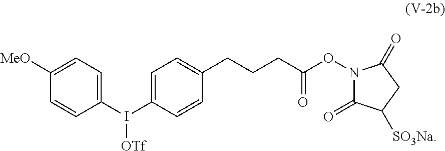

24. A composition comprising the compound of claim 22 and the compound (II-3a): 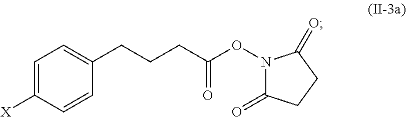
wherein X is selected from the group consisting of I-123, I-124, I-125 and I-131.

25. A composition comprising the compound of claim 23 and the compound (II-3b): 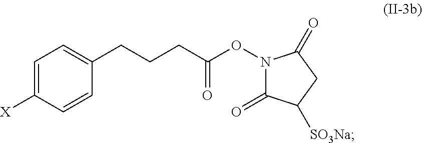
wherein X is selected from the group consisting of I-123, I-124, I-125 and I-131.

26. The compound of claim 1 having the structure of formula (VI): 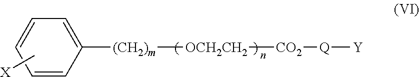
wherein m is 1-6;
n is 0-3;
Q is selected from alkyl, heteroalkyl, carboxyalkyl and carboxyheteroalkyl; and
Y is selected from the group consisting of succinimidyl and sulfosuccinimidyl.

27. (canceled)

28. The compound of claim 1 having the structure of formula (VII): 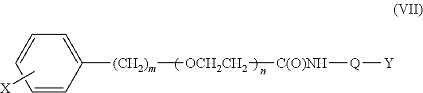
wherein m is 1-6;
n is 0-3;
Q is selected from alkyl, heteroalkyl, carboxyalkyl and carboxyheteroalkyl; and
Y is selected from the group consisting of succinimidyl and sulfosuccinimidyl.

29. (canceled)
